(19) United States
(12) Patent Application Publication
(10) Pub. No.: US 2020/0283502 A1
(43) Pub. Date: Sep. 10, 2020

(54) POSITIVE ALLOSTERIC MODULATORS OF GABA A RECEPTOR

(71) Applicant: PEPTICOM LTD., Jerusalem (IL)

(72) Inventors: Immanuel LERNER, Jerusalem (IL); Amit MICHAELI, Jerusalem (IL)

(21) Appl. No.: 16/649,813

(22) PCT Filed: Sep. 25, 2018
(86) PCT No.: PCT/IB2018/057379

§ 371 Date: Mar. 23, 2020

Related U.S. Application Data
(60) Provisional application No. 62/562,581, filed on Sep. 25, 2017.

(51) Int. Cl. C07K 14/705; C07K 5/097; C07K 5/11; C07K 5/117; G01N 33/94; G01N 33/68

(52) U.S. Cl. CPC C07K 14/70571; C07K 5/0821; C07K 5/1019; A61K 38/00; G01N 33/9426; G01N 33/689; C07K 5/1024

(57) ABSTRACT

The present invention relates to a GABA_{A} receptor-binding peptide comprising an amino acid sequence X₁-X₂-X₃-X₄-X₅, wherein the amino acid residue X₁ is histidine, arginine, threonine, L-cyclohexyl-alanine, 2-flouro-L-phenylalanine or 3-methyl-L-histidine; X₂ is threonine, N-methyl-threonine, proline, leucine, isoleucine or phenylalanine; X₃ is tryptophan, N-methyl-tryptophan, serine, threonine or proline; X₄ is glutamine, proline, lysine, tyrosine, alanine, glycine or absent; and X₅ is lysine, glutamic acid, aspartic acid, threonine, alanine, glycine or absent. In particular, the GABA_{A} receptor-binding peptides of the present invention have amino acid sequences selected from SEQ ID NOs: 1 to 15. These peptides were tested and validated using electrophysiological recordings on the human GABA_{A} receptor comprising of the following subunits α₁β₃γ₂ and used in the preparation of a neuroactive pharmaceutical composition, in improving sperm motility or in labeling of biomolecules.

### TECHNICAL FIELD

The present invention relates to the field of neuroscience, and more particularly, to peptides that modify the activation of the human γ-aminobutyric acid receptor A (GABA_{A} receptor).

### BACKGROUND

GABA is the main inhibitory neurotransmitter in both vertebrate and invertebrate organisms (Gou et al. 2012, Evolution of neurotransmitter gamma-aminobutyric acid, glutamate and their receptors, Dongwuxue Yanjiu. 33(E5-6): E75-81). GABA receptors are divided into two major classes: the GABA_{A} ionotropic C1-channels and the G protein-coupled GABA_{B} receptors. GABA_{A} receptors play a crucial role in the central nervous system (CNS) in homeostasis and pathological conditions, such as anxiety disorder, epilepsy, insomnia, spasticity, aggressive behavior, and other pathophysiological conditions and diseases (Jemberk et al. 2015, GABA Receptors: Pharmacological Potential and Pitfalls, Current Pharmaceutical Design 21, 4943-59). GABA receptors have been linked to physiological activity outside of the nervous system, in roles like modulation of sperm motility and others.

U.S. Pat. No. 6,380,210 B1 describes substituted heteroaryl fused aminoalkyl-imidazole derivatives acting as selective modulators of GABA_{A} receptors and their use in enhancing alertness and treating anxiety, overdoses of benzodiazepine-type drugs, Down syndrome, depression, sleep, seizure and cognitive disorders both in human as well as domestic pets and livestock. U.S. Pat. No. 6,218,547 B1 discloses 1-phenyl-benzimidazole derivatives also acting as GABA_{A} receptor modulators and used to treat the CNS-related disorders, such as anxiety, anesthesia, epilepsy, or convulsions in humans and animals.

U.S. Pat. No. 7,425,556 B1 discloses a number of cinnoline compounds including some selected 4-amino- and 4-oxo-cinnoline-3-carboxamides capable of modulating activity of the GABA_{A} receptor and used as medicaments for treating or preventing an anxiety disorder, cognitive disorder, or mood disorder. US 2005/0101614 A1 describes a number of heterocyclic GABA_{A}-subtype selective receptor modulators selected from substituted derivatives of 7-arylindazole, 7-al-2H-pyrazolo[3,4-c]pyridine, 7-aryl-2H-pyrazolo[4,3-c]pyridine and 7-aryl-2H-pyrazolo[4,3-b]pyridine compounds.

However, none of the prior art publications discloses or suggests the novel short linear peptides of the present invention or suggests their use as CNS depressants. The short linear peptides of the present invention were tested and validated as positive allosteric modulators of the human α₁β₃γ₂ GABA_{A} receptor. The discovered peptides have a strong effect on any physiological/pathological process involving the activity of GABA_{A} receptor, including but not limited to anxiolytic, sedative, and hypnotic effects as well as non-neurological roles such as modulation of sperm activity.

### SUMMARY

One aspect of the present invention provides a GABA_{A} receptor-binding peptide comprising an amino acid sequence:

X₁-X₂-X₃-X₄-X₅,

wherein:
X₁ is histidine, arginine, threonine, L-cyclohexyl-alanine, 2-flouro-L-phenylalanine or 3-methyl-L-histidine;
X₂ is threonine, N-methyl-threonine, proline, leucine, isoleucine or phenylalanine;
X₃ is tryptophan, N-methyl-tryptophan, serine, threonine or proline;
X₄ is glutamine, proline, lysine, tyrosine, alanine, glycine or absent; and
X₅ is lysine, glutamic acid, aspartic acid, threonine, alanine, glycine or absent.

In particular, the GABA_{A} receptor-binding peptide of the present invention has an amino acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and SEQ ID NO:15.

In a certain embodiment, the peptide of the present invention further comprises at least one additional amino acid residue at the N-terminus of the sequence or at the C-terminus of the sequence. In a particular embodiment, the peptide of the present invention further comprises an antigen to a particular antibody at the N-terminus of the sequence or at the C-terminus of the sequence. In another embodiment, the peptide of the present invention further comprises a fluorescent or non-fluorescent labeling molecule at the N-terminus of the sequence or at the C-terminus of the sequence. In still another embodiment, said labeling molecule is radioactive or comprising an electron-spin resonance moiety.

In another aspect, the peptide of the present invention is used in the preparation of a neuroactive pharmaceutical composition, in improving sperm motility or in labeling of biomolecules.

All the compounds of the present invention were tested and validated using electrophysiological recordings on the human GABA_{A} receptor comprising of the following subunits α₁β₃γ₂.

Various embodiments may allow various benefits, and may be used in conjunction with various applications. The details of one or more embodiments are set forth in the accompanying figures and the description below. Other features, objects and advantages of the described techniques will be apparent from the description and drawings and from the claims.

### BRIEF DESCRIPTION OF THE DRAWINGS

Disclosed aspects of the present invention will be understood and appreciated more fully from the following detailed description taken in conjunction with the appended figures. The drawings included and described herein are schematic and are not limiting the scope of the disclosure.

FIG. 1 shows selective potentiation of human GABA receptor-mediated Cl⁻ current by the SEQ ID NO:1 peptide (HTWQE). The human GABA receptor (subunits α₁β₃γ₂) was expressed in HEK293 cells in manual whole-cell patch-clamp settings.

FIG. 2 shows selective potentiation of human GABA receptor-mediated Cl⁻ current by the SEQ ID NO:2 peptide (L-cyclohexylalanine-TWQE). The human GABA receptor (subunits α₁β₃γ₂) was expressed in HEK293 cells in manual whole-cell patch-clamp settings.

FIG. 3 shows selective potentiation of human GABA receptor-mediated Cl⁻ current by the SEQ ID NO:3 peptide (3-methyl-L-histidine-N-methyl-threonine-WQE). The human GABA receptor (subunits α₁β₃γ₂) was expressed in HEK293 cells in manual whole-cell patch-clamp settings.

FIG. 4 shows selective potentiation of human GABA receptor-mediated Cl⁻ current by the SEQ ID NO:4 peptide (3-methyl-L-histidine-N-methyl-threonine-N-methyl tryptophan-QE). The human GABA receptor (subunits α₁β₃γ₂) was expressed in HEK293 cells in manual whole-cell patch-clamp settings.

FIG. 5 shows selective potentiation of human GABA receptor-mediated Cl⁻ current by the SEQ ID NO:5 peptide (2-flouro-L-phenylalanine-TWQE). The human GABA receptor (subunits α₁β₃γ₂) was expressed in HEK293 cells in manual whole-cell patch-clamp settings.

FIG. 6 shows selective potentiation of human GABA receptor-mediated Cl⁻ current by the SEQ ID NO:6 peptide (HTWKK). The human GABA receptor (subunits α₁β₃γ₂) was expressed in HEK293 cells in manual whole-cell patch-clamp settings.

FIG. 7 shows selective potentiation of human GABA receptor-mediated Cl⁻ current by the SEQ ID NO:7 peptide (HTWYE). The human GABA receptor (subunits α₁β₃γ₂) was expressed in HEK293 cells in manual whole-cell patch-clamp settings.

FIG. 8 shows selective potentiation of human GABA receptor-mediated Cl⁻ current by the SEQ ID NO:8 peptide (HPPAT). The human GABA receptor (subunits α₁β₃γ₂) was expressed in HEK293 cells in manual whole-cell patch-clamp settings.

FIG. 9 shows selective potentiation of human GABA receptor-mediated Cl⁻ current by the SEQ ID NO:9 peptide (HIS-NH₂). The human GABA receptor (subunits α₁β₃γ₂) was expressed in HEK293 cells in manual whole-cell patch-clamp settings.

FIG. 10 shows selective potentiation of human GABA receptor-mediated Cl⁻ current by the SEQ ID NO:10 peptide (RFHS). The human GABA receptor (subunits α₁β₃γ₂) was expressed in HEK293 cells in manual whole-cell patch-clamp settings.

FIG. 11 shows selective potentiation of human GABA receptor-mediated Cl⁻ current by the SEQ ID NO:11 peptide (TESKG-NH₂). The human GABA receptor (subunits α₁β₃γ₂) was expressed in HEK293 cells in manual whole-cell patch-clamp settings.

FIG. 12 shows selective potentiation of human GABA receptor-mediated Cl⁻ current by the SEQ ID NO:12 peptide (HTTGD). The human GABA receptor (subunits α₁β₃γ₂) was expressed in HEK293 cells in manual whole-cell patch-clamp settings.

FIG. 13 shows selective potentiation of human GABA receptor-mediated Cl⁻ current by the SEQ ID NO:13 peptide (RTWGE). The human GABA receptor (subunits α₁β₃γ₂) was expressed in HEK293 cells in manual whole-cell patch-clamp settings.

FIG. 14 shows selective potentiation of human GABA receptor-mediated Cl⁻ current by the SEQ ID NO:14 peptide (HTWP). The human GABA receptor (subunits α₁β₃γ₂) was expressed in HEK293 cells in manual whole-cell patch-clamp settings.

FIG. 15 shows selective potentiation of human GABA receptor-mediated Cl⁻ current by the SEQ ID NO:15 peptide (HPWQ). The human GABA receptor (subunits α₁β₃γ₂) was expressed in HEK293 cells in manual whole-cell patch-clamp settings.

FIG. 16a shows calculated binding energy contributions for the SEQ ID NO:1 peptide having most of the binding energy contributions by the first three amino-acids from the N-terminus.

FIG. 16b shows calculated binding energy contributions for the SEQ ID NO:10 peptide having relatively evenly distributed binding free energy contributions.

FIG. 17 shows the effect of the SEQ ID NO:1 peptide on the percentage of motile mouse sperm cells in comparison to control peptide and DMSO solvent.

FIG. 18 shows the effect of the SEQ ID NO:1 peptide on acrosome release of motile mouse sperm cells in comparison to control peptide and DMSO solvent.

### DETAILED DESCRIPTION

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The term “comprising”, used in the claims, is “open ended” and means the elements recited, or their equivalent in structure or function, plus any other element or elements which are not recited. It should not be interpreted as being restricted to the means listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a device comprising x and z” should not be limited to devices consisting only of components x and z. Also, the scope of the expression “a method comprising the steps x and z” should not be limited to methods consisting only of these steps.

Unless specifically stated, as used herein, the term “about” is understood as within a range of normal tolerance in the art, for example within two standard deviations of the mean. In one embodiment, the term “about” means within 10% of the reported numerical value of the number with which it is being used, preferably within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value. In other embodiments, the term “about” can mean a higher tolerance of variation depending on for instance the experimental technique used. Said variations of a specified value are understood by the skilled person and are within the context of the present invention. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges, for example from 1-3, from 2-4, and from 3-5, as well as 1, 2, 3, 4, 5, or 6, individually. This same principle applies to ranges reciting only one numerical value as a minimum or a maximum. Unless otherwise clear from context, all numerical values provided herein are modified by the term “about”. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any and all combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached to”, “connected to”, “coupled with”, “contacting”, etc., another element, it can be directly on, attached to, connected to, coupled with or contacting the other element or intervening elements may also be present. In contrast, when an element is referred to as being, for example, “directly on”, “directly attached to”, “directly connected to”, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

In one aspect, the present invention provides a GABA_{A} receptor-binding peptide comprising an amino acid sequence:

X₁-X₂-X₃-X₄-X₅,

wherein:
X₁ is histidine, arginine, threonine, L-cyclohexyl-alanine, 2-flouro-L-phenylalanine or 3-methyl-L-histidine;
X₂ is threonine, N-methyl-threonine, proline, leucine, isoleucine or phenylalanine; X₃ is tryptophan, N-methyl-tryptophan, serine, threonine or proline;
X₄ is glutamine, proline, lysine, tyrosine, alanine, glycine or absent; and
X₅ is lysine, glutamic acid, aspartic acid, threonine, alanine, glycine or absent.

In a certain embodiment, X₁ is histidine, 3-methyl-L-histidine or arginine, in particular X₁ is histidine. In a further embodiment, X₂ is threonine, N-methyl-threonine or proline, in particular threonine. In yet further embodiment, X₃ is tryptophan, N-methyl-tryptophan or serine, in particular tryptophan. In another embodiment, X₄ is glutamine, lysine or glycine, in particular glutamine. In still another embodiment, X₅ is glutamic acid. In one of the embodiments, X₄ is absent resulting in three-amino acids peptides, or X₅ is absent resulting in four-amino acid peptides. In a specific embodiment, the N-terminus of the peptide of the present invention can be acetylated.

In a particular embodiment, the GABA_{A} receptor-binding peptide of the present invention has an amino acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and SEQ ID NO:15. These sequences are shown in the following table:

| SEQ ID | | | |
|---|---|---|---|
| NO. | FASTA Sequence | Structure | Activity |
| 1 | HTWQE | His—Thr—Trp—Gln—Glu | Positive allosteric modulator |
| 2 | (L-cyclohexyl-alanine)- TWQE | 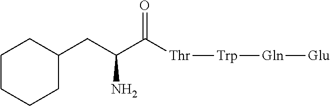 | Positive allosteric modulator |
| 3 | (3-methyl-L-histidine)- (N-methyl-threonine)- WQE | 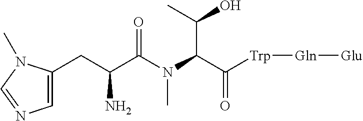 | Positive allosteric modulator |
| 4 | (3-methyl-L-histidine)- (N-methyl-threonine)- (N-methyl-tryptophan)- QE | 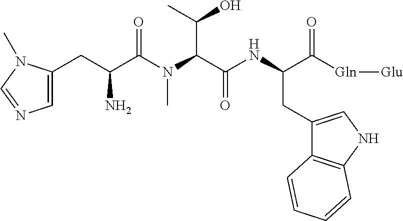 | Positive allosteric modulator |
| 5 | (2-flouro-L-phenyl- alanine)-TWQE | 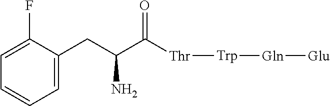 | Positive allosteric modulator |
| 6 | HTWKK | His—Thr—Trp—Lys—Lys | Positive allosteric modulator |
| 7 | HTWYE | His—Thr—Trp—Tyr—Glu | Positive allosteric modulator |
| 8 | HPPAT | His—Pro—Pro—Ala—Thr | Positive allosteric modulator |
| 9 | HIS—NH₂ | His—Ile—Ser—NH₂ | Positive allosteric modulator |
| 10 | RFHS | Arg—Phe—His—Ser | Positive allosteric modulator |
| 11 | TESKG—NH₂ | Thr—Glu—Ser—Lys—Gly—NH₂ | Positive allosteric modulator |
| 12 | HTTGD | His—Thr—Thr—Gly—Asp | Positive allosteric modulator |
| 13 | RTWGE | Arg—Thr—Trp—Gly—Glu | Positive allosteric His—Thr—Trp—Gln—Glu modulator |
| 14 | HTWP | His—Thr—Trp—Pro | Positive allosteric modulator |
| 15 | HPWQ | His—Pro—Trp—Gln | Positive allosteric modulator |

The amino acid sequences of the human GABA_{A} receptor-modulating peptides recited above are from their N-terminus to their C-terminus. The peptides having the above listed SEQ ID NOs 1-15 of the present invention were computationally designed to bind the GABA_{A} receptor, either as a partial peptide, or as a part of a larger polypeptide. These peptides are experimentally shown to modulate the GABA_{A} receptor.

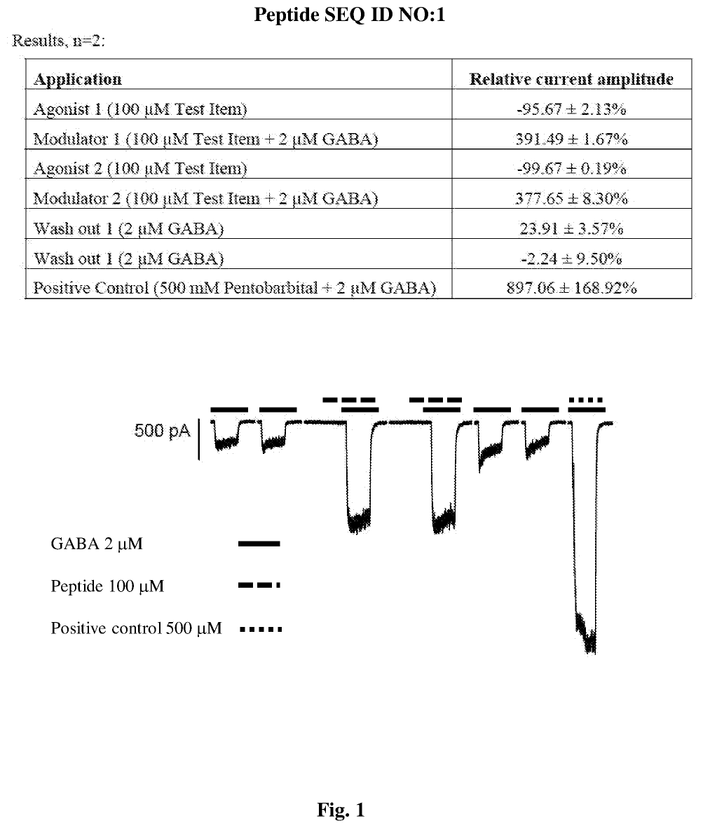

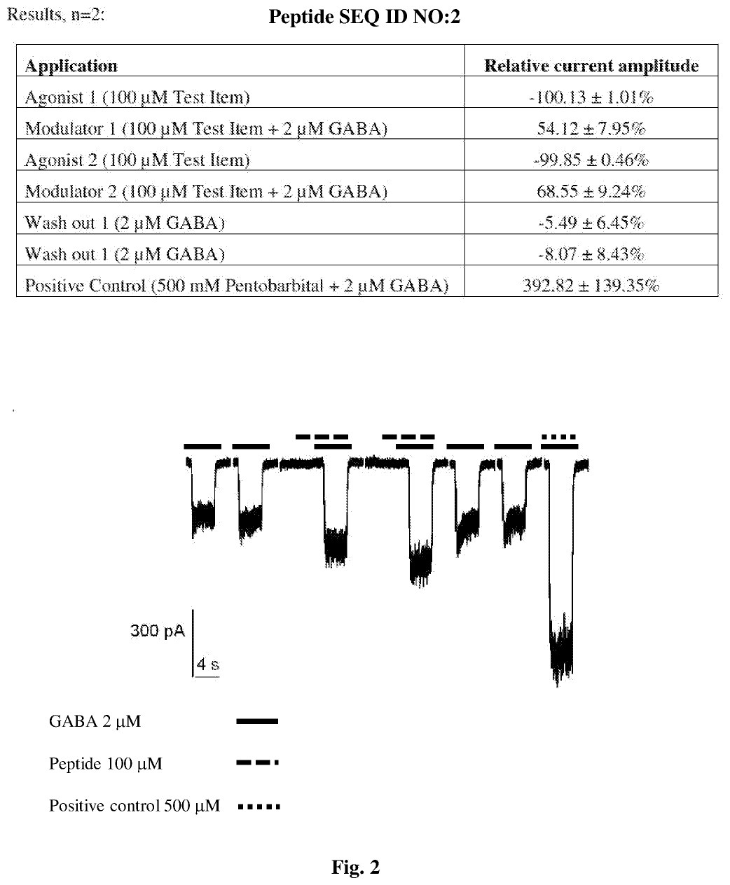

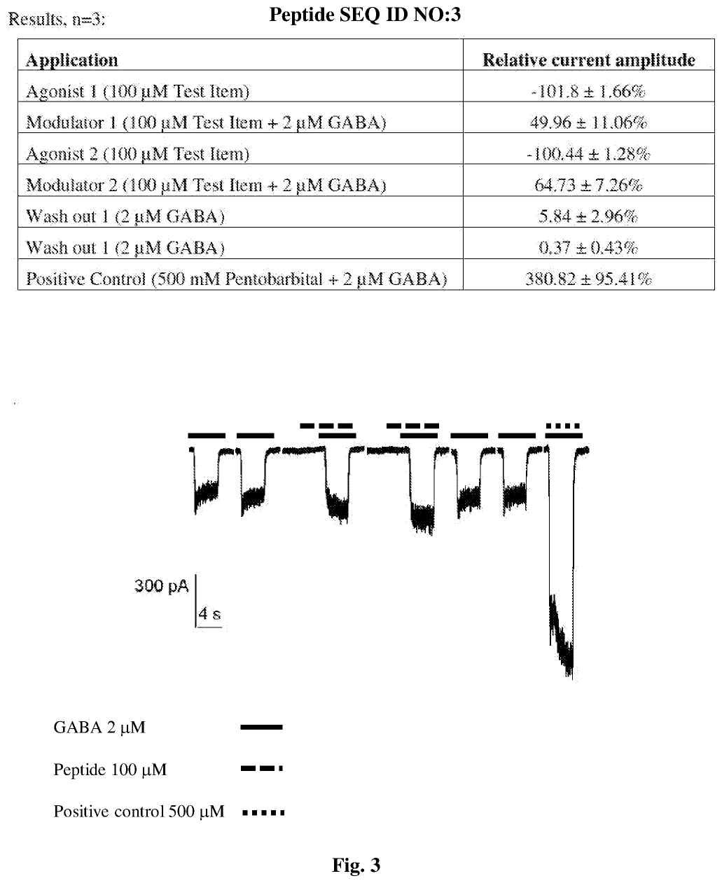

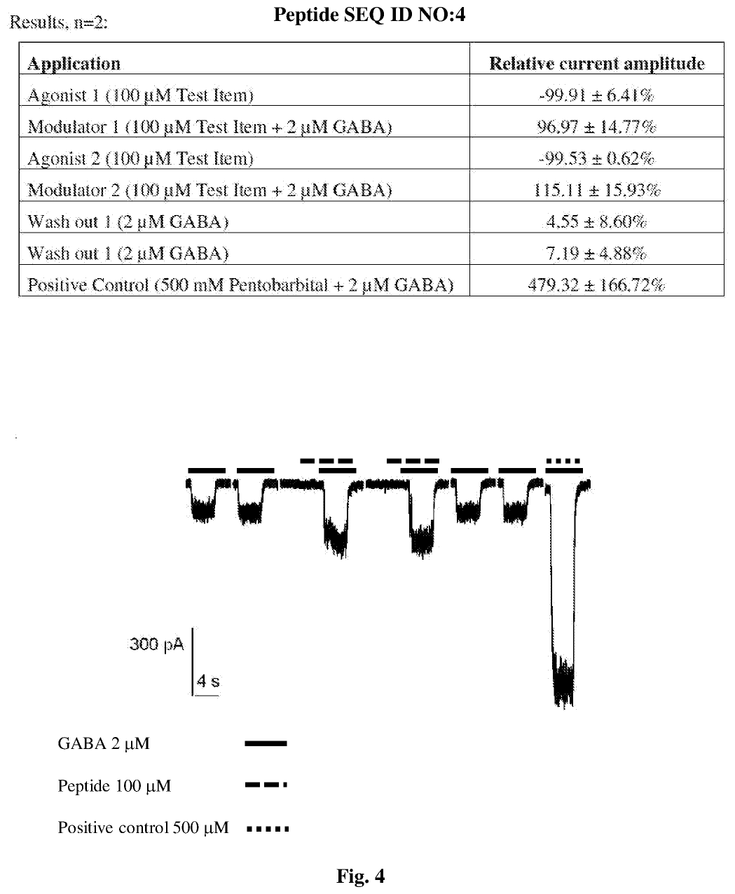

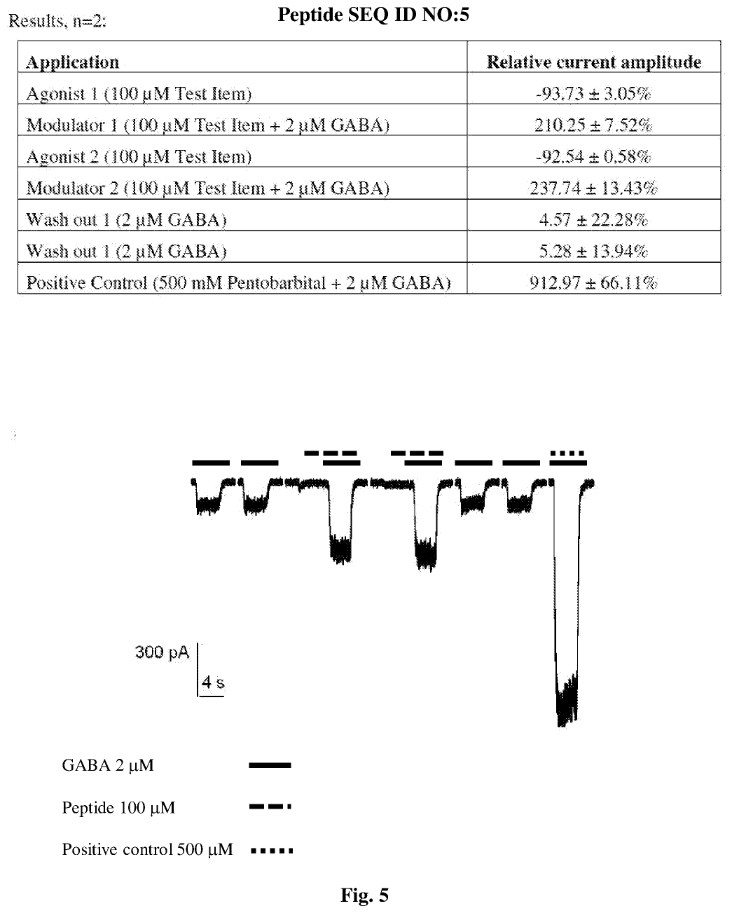

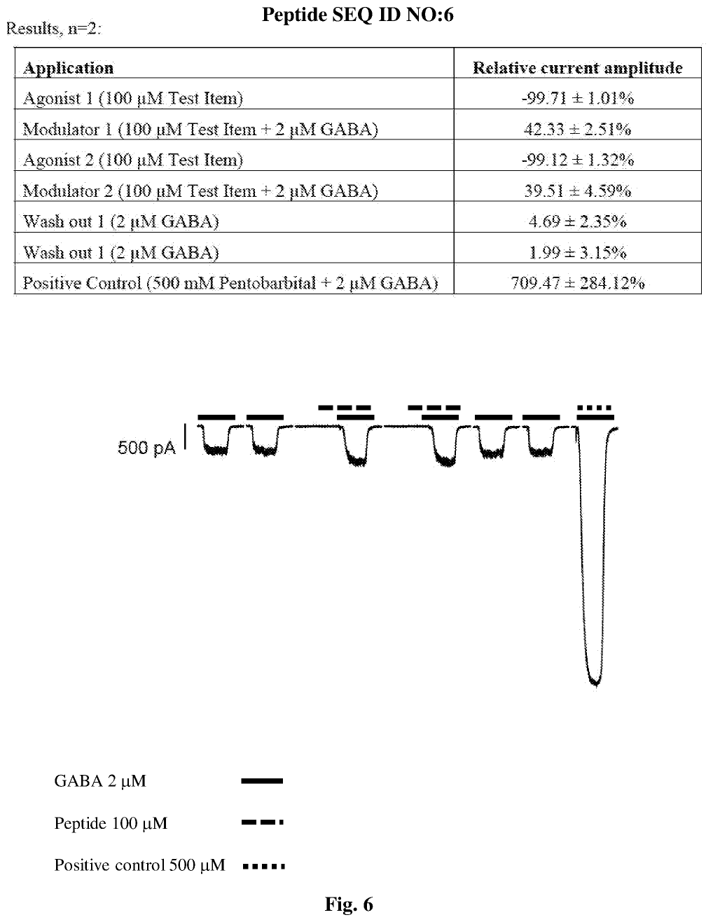

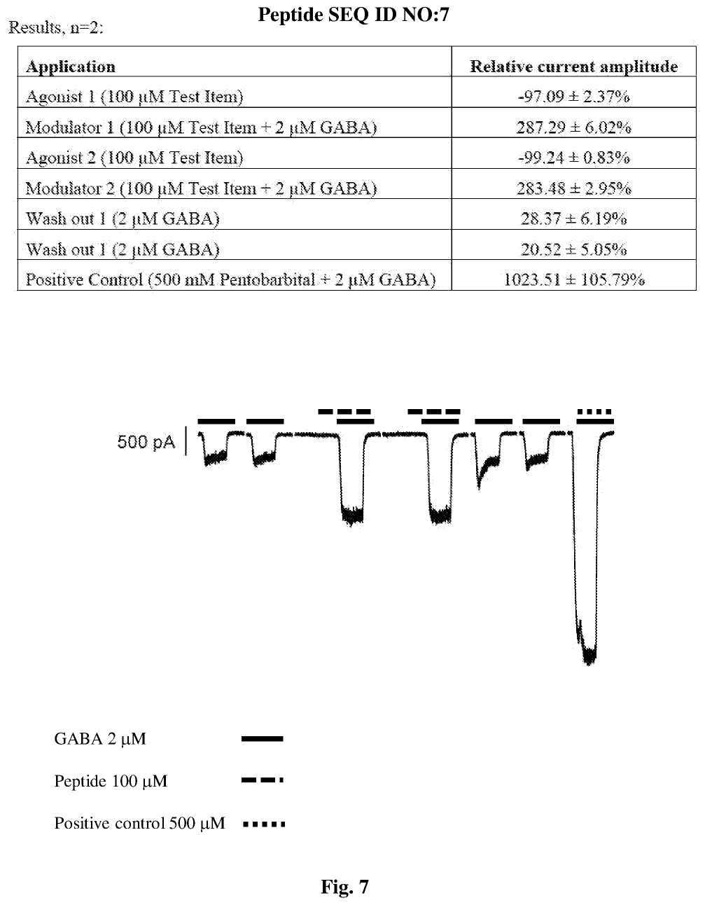

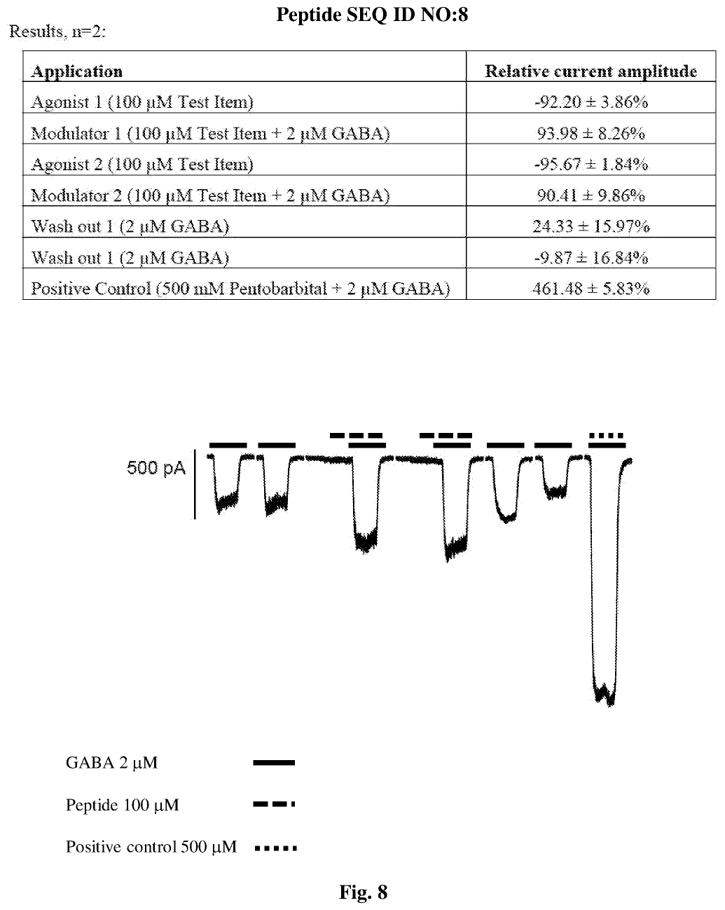

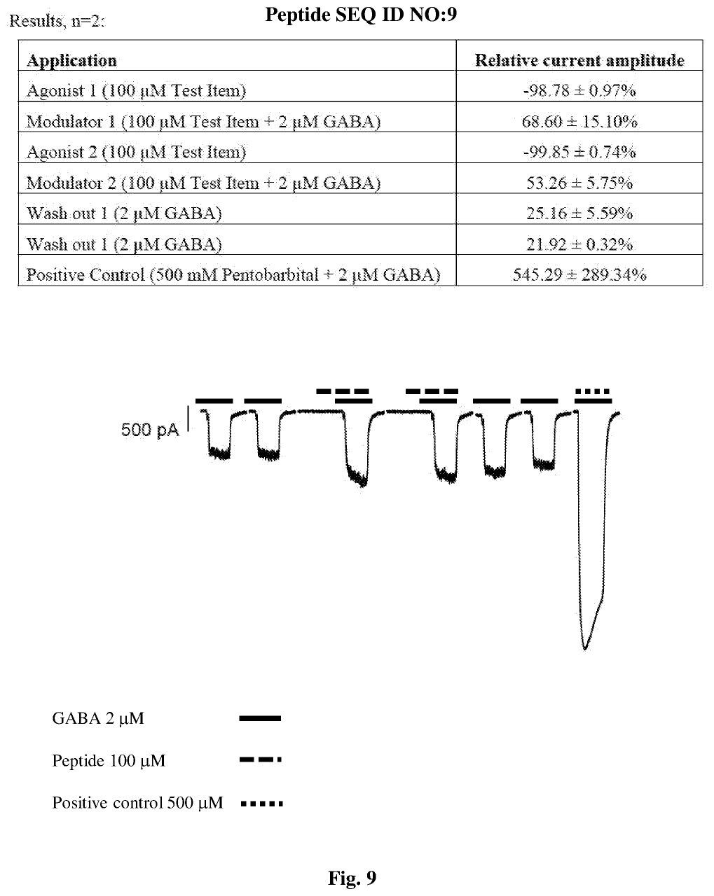

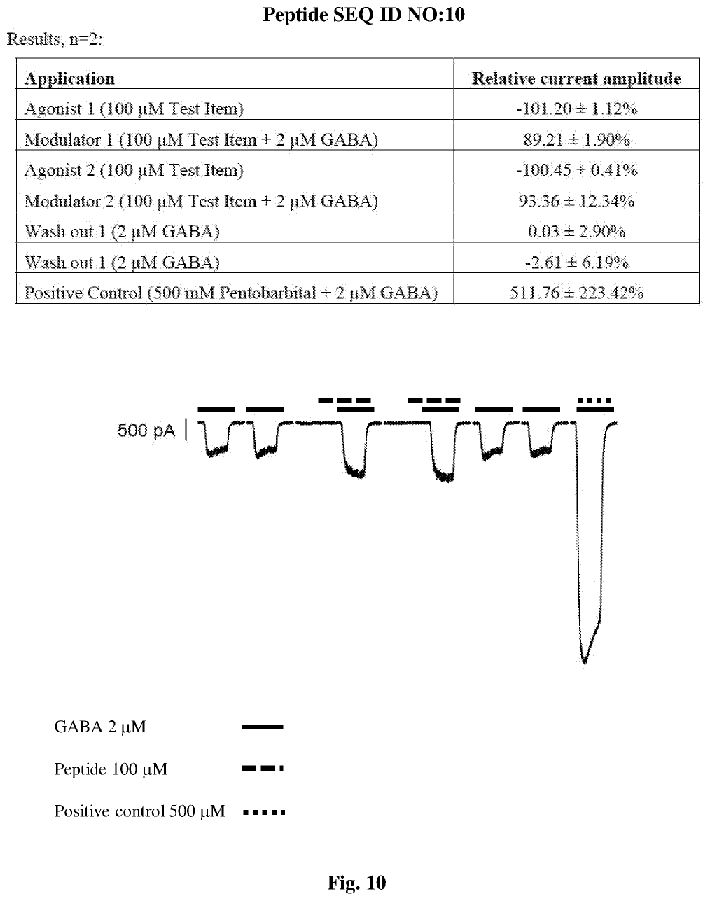

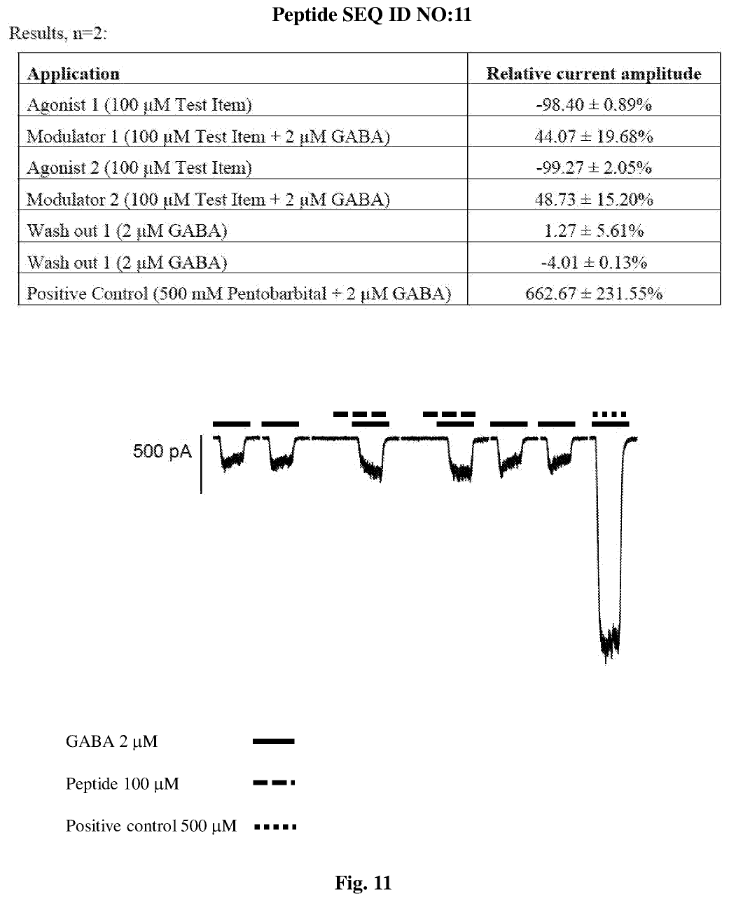

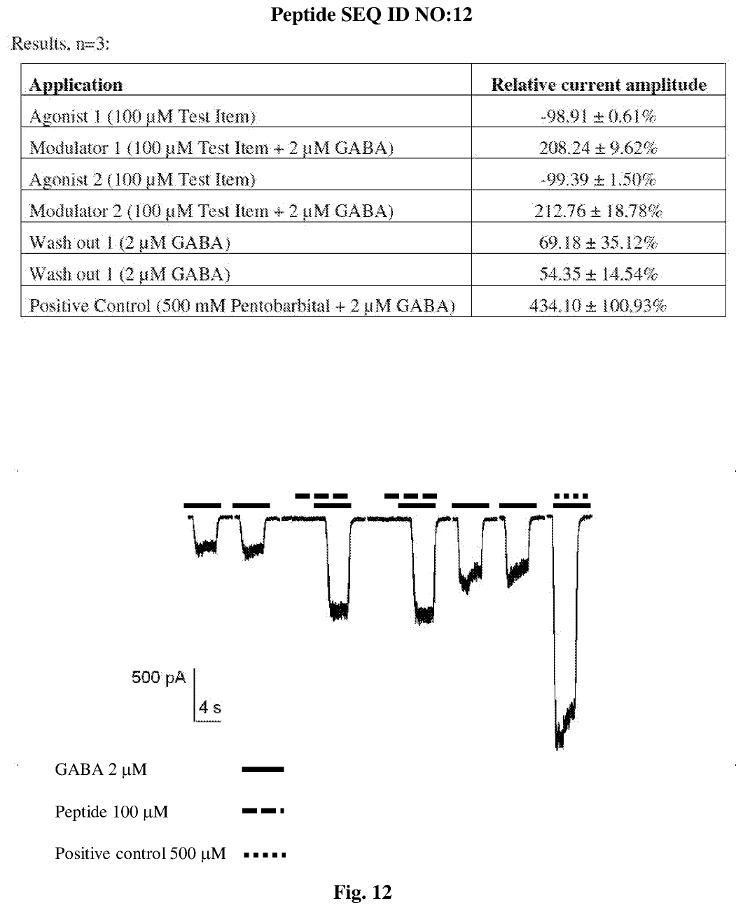

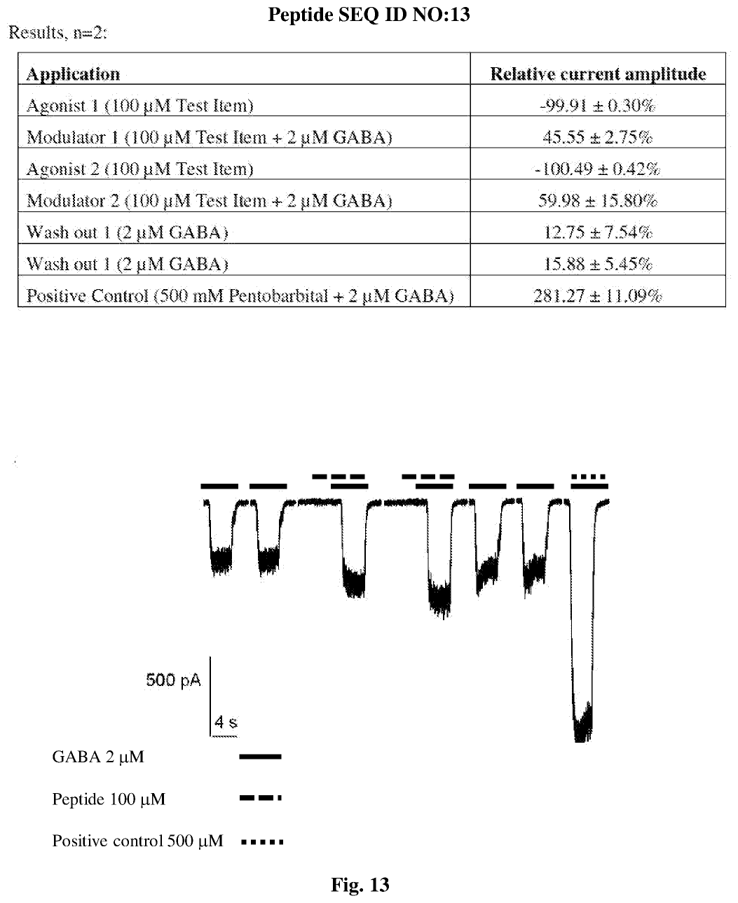

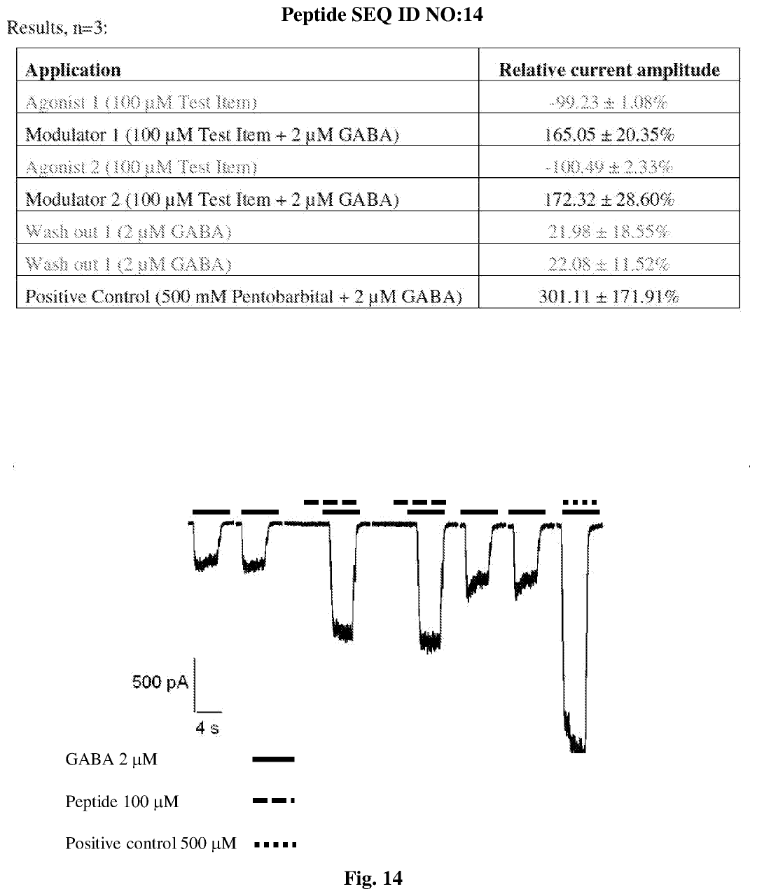

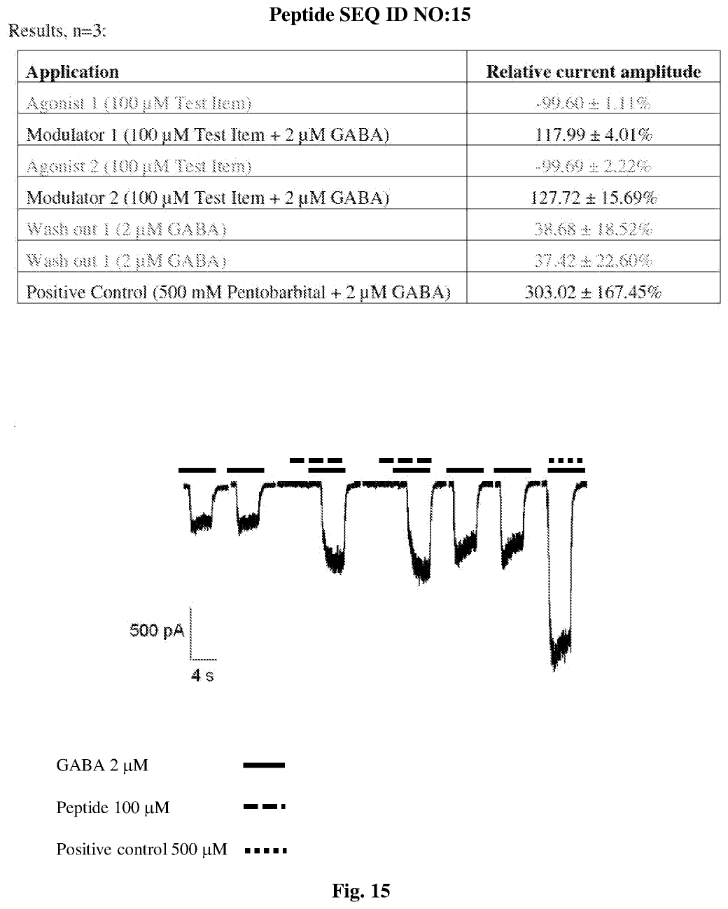

The peptides of the present invention are capable of activating, inhibiting or modulating the GABA_{A} receptor. These peptides were derived in-silico and tested in-vitro in cell cultures. FIGS. 1-15 demonstrate the selective potentiation of the human GABA receptor-mediated Cl⁻ current by the instant peptides having SEQ ID NOs 1-15. The human GABA receptor (subunits α₁β₃γ₂) used in these experiments was expressed in HEK293 cells in manual whole-cell patch-clamp settings.

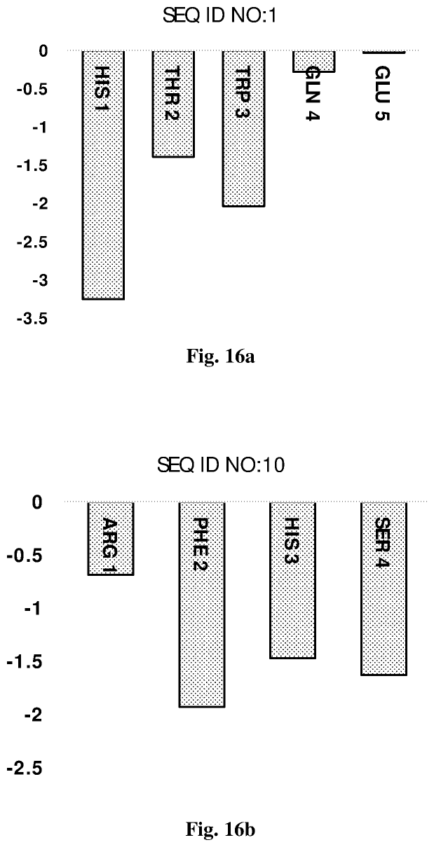

The peptides of the present invention were designed to specifically bind the mammalian α₁β₃γ₂ GABA_{A} channel's γ-aminobutyric (GABA) binding pocket in a similar manner as GABA, with the exception of SEQ ID NOs 10 and 11. Reference is now made to FIGS. 16a and 16b showing the calculated binding energy contributions for the SEQ ID NO:1 and SEQ ID NO:10 peptides, respectively. While the SEQ ID NO:1 has most of the binding energy contributions by the first three amino-acids from the N-terminus, the SEQ ID NO:10 peptide has relatively evenly distributed binding free energy contributions. The SEQ ID NO:11 peptide is similar in its activity to the SEQ ID NO:10 peptide. All other peptides, except SEQ ID NO:11, follow the same general activity pattern as the SEQ ID NO:1 peptide.

The residue-specific binding energy contributions shown in FIGS. 16a and 16b suggested the specific design of a peptide having sequence X₁-X₂-X₃-X₄-X₅, wherein the amino acid residue X₁=H, R, T, L-cyclohexane, 2-flouro-L-phenylalanine or 3-methyl-L-histidine; X₂=T, P, L, I or F; X₃=W, S, T or P; X₄=Q, P, K, Y, A or G; and X₅=K, E, D, T, A or G. The amino acid residues X₂ and X₃ are compatible with the N-methylated backbone for any of the amino acids detailed above. The amino acid residues X₄ and X₅ are found (from calculation) to contribute little to binding (see FIGS. 16a and 16b). Variants, such as SEQ ID NO:9 may exist without both X₄ and X₅, while SEQ ID NO:10, SEQ ID NO:14 and SEQ ID NO:15 do not contain X₅. Similarly, additional amino acids can be added to the C-terminus of these sequences while retaining their activity. Any of these combinations may be considered a candidate for a GABA_{A} channel binding peptide. Some specific combinations may be selected with respect to delivery considerations of the peptide to the target tissue, e.g., with respect to the peptide's solubility and biological interactions that may be determined experimentally along the lines exemplified herein for specific peptide examples.

In another aspect, these peptides of the present invention are used for the preparation of neuroactive or psychoactive compositions, such as anti-depressants, anti-addictive or anti-epileptic drugs, or any other medical compositions, which are capable of exhibiting the GABA_{A} receptor modulation.

Specific combinations of the peptides of the present invention may be selected with respect to delivery considerations of the peptide to the target tissue, e.g., with respect to the peptide's solubility and biological interactions that may be determined experimentally along the lines exemplified herein for specific peptide examples.

In certain embodiments, possible applications of the peptides of the present invention or their molecular derivatives are in the pharmaceutical industry as drugs for any relevant clinical indication with a need to modify GABA_{A} receptor activity. They may also be used in a wide variety of clinical applications, as well as in diagnostics and imaging applications. Non-limiting examples of using these peptides comprise protection from anti-depressants and anti-addictive indications. They may be also used for fluorescent or non-fluorescent biolabeling in the process of modulating and binding the GABA_{A} receptor for experimental use, in in-vitro or in-vivo, and as specific inhibitors for basic research (in neuroscience).

### Examples

### Experimental Procedure for Discovery and Calculation of Peptide Binding

It has been experimentally found that the peptides of the present invention can be used to significantly improve sperm motility. For motility experiments, murine sperm was collected in a modified Whitten's medium (MW; 22 mM HEPES, 1.2 mM MgCl₂, 100 mM NaCl, 4.7 mM KCl, 1 mM pyruvic acid, 4.8 mM lactic acid hemi-Ca²⁺ salt, pH 7.35). All steps of collection and washing were performed at 37° C. After the initial washing, but prior to experimental incubations, motility assessment was carried out. Assessment of motility was done under capacitating conditions using media supplemented with both 10 mM NaHCO₃ and 1 mM 2-OHCD as capacitating conditions (pH=7.35). Motility percentage of sperm under different conditions was assessed using video capture (the video is available upon request).

Acrosome release (AR) was assessed under capacitating conditions, first sperm was collected in a modified Whitten's medium (MW; 22 mM HEPES, 1.2 mM MgCl₂, 100 mM NaCl, 4.7 mM KCl, 1 mM pyruvic acid, 4.8 mM lactic acid hemi-Ca²⁺ salt, pH 7.35). Capacitation was triggered for different experimental groups via supplementation with both 10 mM NaHCO₃ and 1 mM 2-OHCD as capacitating conditions (pH=7.35). Sperm was then processed for Coomassie assessment of AR.

### Calculation of the Binding Energy Contributions

The binding energy contributions were calculated using an ab initio algorithm that takes into account molecular mechanics force-fields in 3D (three dimensional) space and at a 1 Å resolution. The binding energy contributions were calculated using the Assisted Model Building with Energy Refinement (AMBER) (Cornell 1995, A Second Generation Force Field for the Simulation of Proteins, Nucleic Acids, and Organic Molecules. Journal of the American Chemical Society 117, 5179-97). It was force-field with the Generalized-Born/Surface Area (GB/SA) solvation model, and was already effectively applied to other fields as well (Froese et al. 2015, Structural basis of glycogen branching enzyme deficiency and pharmacologic rescue by rational peptide design, Human Molecular Genetics 24(20), 5667-5676). The obtained data on the binding energy contributions can be used to design modified peptides, e.g., incorporate SEQ ID NOs: 1-15 into larger peptides or modify the sequences while maintaining the overall negative binding energy, as well as to design peptide mimetics and/or small molecules.

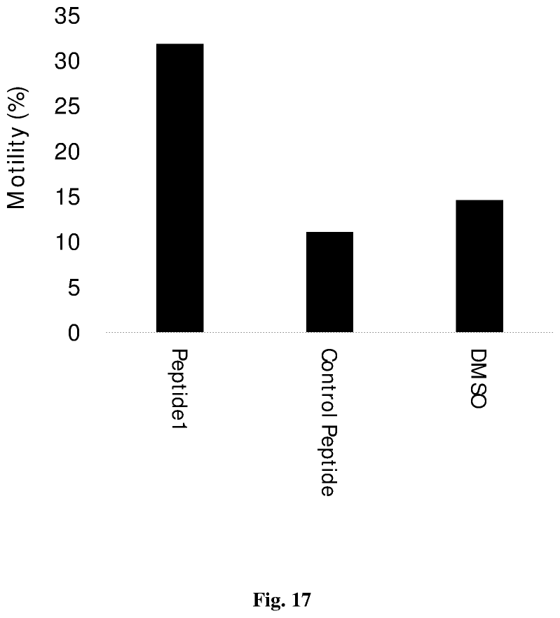

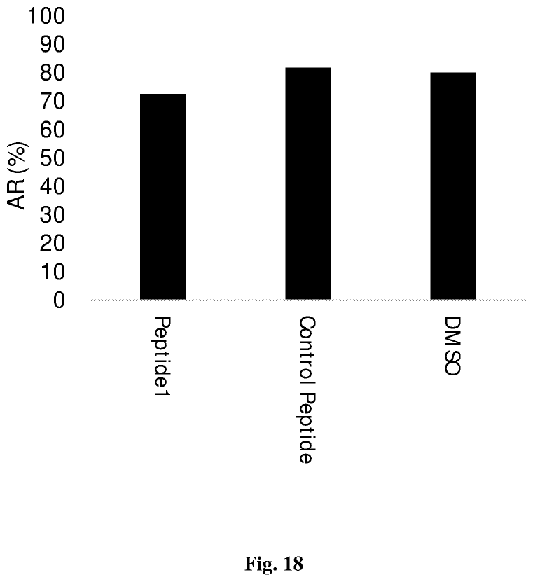

FIG. 17 shows the effect of the SEQ ID NO:1 peptide on the percentage of motile mouse sperm cells in comparison to a control peptide and to DMSO solvent, whereas FIG. 18 shows the effect of the peptide of the on acrosome release of motile mouse sperm cells in comparison to a control peptide and DMSO solvent and predicted binding energy contributions for each amino acid in the peptides of SEQ ID NO:1 and SEQ ID NO:10. These peptides exhibit exemplary binding to GABA_{A} for all other peptides of the present invention, which are predicted (from calculation) for binding via GABA_{A} receptor, according to the embodiments of the present invention. The lower the individual amino acid binding energy contribution is, the more essential it is for the peptide binding (the efficiency of which is determined by the sum of the binding energy contributions).

While certain features of the present application have been illustrated and described herein, many modifications, substitutions, changes, and equivalents will be apparent to those of ordinary skill in the art. It is, therefore, to be understood that the appended claims are intended to cover all such modifications and changes as fall within the true spirit of the present application.

1-20. (canceled)

21. A GABA_{A} receptor-binding peptide comprising an amino acid sequence:
X₁-X₂-X₃-X₄-X₅,
wherein:
X₁ is histidine, arginine, threonine, L-cyclohexyl-alanine, 2-flouro-L-phenylalanine or 3-methyl-L-histidine;
X₂ is threonine, N-methyl-threonine, proline, leucine, isoleucine or phenylalanine;
X₃ is tryptophan, N-methyl-tryptophan, serine, threonine or proline;
X₄ is glutamine, proline, lysine, tyrosine, alanine, glycine or absent; and
X₅ is lysine, glutamic acid, aspartic acid, threonine, alanine, glycine or absent.

22. The peptide of claim 21, wherein X₁ is histidine, 3-methyl-L-histidine or arginine.

23. The peptide of claim 22, wherein X₁ is histidine.

24. The peptide of claim 21, wherein X₂ is threonine, N-methyl-threonine or proline.

25. The peptide of claim 24, wherein X₂ is threonine.

26. The peptide of claim 21, wherein X₃ is tryptophan, N-methyl-tryptophan or serine.

27. The peptide of claim 26, wherein X₃ is tryptophan.

28. The peptide of claim 21, wherein X₄ is glutamine, lysine or glycine.

29. The peptide of claim 28, wherein X₄ is glutamine.

30. The peptide of claim 21, wherein X₅ is glutamic acid.

31. The peptide of claim 21, wherein X₄ is absent.

32. The peptide of claim 21, wherein X₅ is absent.

33. The peptide of claim 21 having an amino acid sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and SEQ ID NO:15.

34. The peptide of claim 21 further comprising at least one additional amino acid residue at the N-terminus of the sequence or at the C-terminus of the sequence.

35. The peptide of claim 21 further comprising an antigen to a particular antibody at the N-terminus of the sequence or at the C-terminus of the sequence.

36. The peptide of claim 21 further comprising a fluorescent or non-fluorescent labeling molecule at the N-terminus of the sequence or at the C-terminus of the sequence.

37. The peptide of claim 36, wherein said labeling molecule is radioactive or comprising an electron-spin resonance moiety.

38. The peptide of claim 21 for use in the preparation of a neuroactive pharmaceutical composition.

39. The peptide of claim 21 for use in improving sperm motility.

40. The peptide of claim 21 for use in labeling of biomolecules.
